(19) United States
(12) Patent Application Publication
(10) Pub. No.: US 2007/0287924 A1
(43) Pub. Date: Dec. 13, 2007

(54) Device And Method for Operating A Patient Monitor

(76) Inventors: Raymond Glocker, Aschaffenburg (DE); Haldun Ozdemir, Fatih-Ankara (TR)

Correspondence Address: KNOBBE MARTENS OLSON & BEAR LLP, 2040 MAIN STREET, FOURTEENTH FLOOR, IRVINE, CA, 92614, US

(21) Appl. No.: 11/547,964

(22) PCT Filed: Jun. 2, 2005
(86) PCT No.: PCT/EP05/05950

§ 371 Date: May 24, 2007

(30) Foreign Application Priority Data
Jun. 2, 2004 (DE) 10 2004 027 044.9

(51) Int. Cl. A61B 5/02

(52) U.S. Cl. 600/493

(57) ABSTRACT

To reduce the susceptibility to interference of a measured signal for invasive blood pressure which has been provided according to standard during patient monitoring, the invention proposes a lemon for transmitting a measured signal for the blood pressure of a patient from a pressure sensor (2) to a display or recording appliance, containing at least a first transmission apparatus (transponder 1) having at least an analog/digital converter for converting the analog measured signal into digital measured data, having at least a first microprocessor (12) for logically combining the digital measured data with test data and having at least an output stage (13) for transmitting the logically combined digital signal comprising measured data and test data, a second transmission apparatus (transceiver 6) having a second microprocessor (15) for separately processing measured data and test data, having a comparison apparatus for comparing the test data with reference data and having an output stage (18) for providing the measured data or the measured signal for a display or recording appliance, and a transmission link (5) between the transponder (1) and the transceiver (6), and also a method.

The invention relates to a device and a method for operating a patient monitor, particularly with an apparatus for measuring the blood pressure of a patient.

Generally in intensive medicine, but particularly when performing operations, vital parameters for the state of the patient are detected by measurement and are displayed and recorded over a certain period using a display or recording appliance, known as a patient monitor or monitor for short. In addition, a patient monitor of this kind comprises various evaluation functions, so that an alarm signal can be output if monitored parameters are abnormal or are changing unusually, so that, in particular, life-saving measures can be taken by the medical staff.

A prerequisite for automatic monitoring of the vital parameters is naturally that the relevant measured signals are available. In addition, a patient monitor of this kind cannot operate with any greater reliability than the reliability of the measured signals.

Besides the display of the electrocardiogram (ECG) on the patient monitor, the display of the invasive blood pressure is the second most frequent display of a vital parameter on the patient monitor. The invasive blood pressure can be and is usually measured extracorporeally, statically using the principle of communicating columns coupled to the blood pressure on the distal tip of a catheter, and dynamically using the pressure wave propagating in the column, which is usually filled with physiological sodium chloride solution (0.8%), slightly retarded with an extracorporeal pressure sensor, known as a transducer, which is mounted on the infusion stand at the level of the right atrium. A transducer of this kind is either in the form of a disposable transducer with an internal volume which has a fluid connection to the column of liquid coupled to the blood circulation, or is in the form of a reusable transducer which is connected to the fluid system by means of a clip-on pressure dome, for example. A connection of this kind can also be made, particularly advantageously, directly using a fluid column of an applied infusion. Such transducers and pressure domes are described, by way of example, in WO 99/37983, WO 02/03854, or DE 103 21 099 (subsequently published), and also in the other documents cited therein. DE 202 02 131 U1 discloses the process of using a transponder for wirelessly transmitting various biometric data, including the blood pressure.

Alternatively, the invasive blood pressure can be measured using a tip transducer at the tip of a catheter which has been introduced. A measurement obtained through this means is much more accurate in the dynamic range, since it is not attenuated by means of the tube system. However, a measurement of this kind is very expensive and hence is limited, for cost reasons, to a very small number of applications (a few thousand per annum worldwide), based on the very large number of invasive blood pressure measurements (over 20 million times per annum worldwide).

During operations on large vessels or even on the heart, there may quickly be a loss of blood which does not become visible for an instant in the field of operation. This also applies to operations which are performed close to large vessels and/or which last a very long time, for example inserting an artificial hip joint, or other extensive orthopedic operations. Checking the blood pressure is therefore an important characteristic feature for monitoring the vital functions.

The provision of the measured signal for the invasive blood pressure and also the requirements in the event of interference in the signal when monitoring using patient monitors are stipulated in DIN EN 60601-2-34 and DIN EN 60601-2-49, accordingly IEC 60601-2-34 and IEC 60601-2-49 internationally. On this basis, the measured signal voltage can be derived from a voltage provided by the patient monitor and must be 5 μV/V/mmHg (5 microvolts per volt supply voltage and pressure in the unit millimeters of mercury). At a typical supply voltage of 5 V, the measured signal is then 25 μV/mmHg. As the measuring cell, the normal disposable transducer or the reusable transducer with a dome has a piezoresistive chip, usually in the size 2 mm×2 mm, whose diaphragm distorts under applied pressure. The distortion is measured using a Wheatstone bridge circuit. The Wheatstone bridge is operated typically with a supply voltage of 5 V and typically with a supply current of 15 mA from the patient monitor using the patient monitor/pressure transducer connecting cable.

So that the standard's required signal voltage of 5 μV/V/mmHg, that is to say 25 μV/mmHg at a supply voltage of 5 V, is obtained the chip, which usually delivers a signal voltage of between 10 and 15 μV/V/mmHg on account of the production tolerances, is trimmed down to the standard's specified 5 μV/V/mmHg using a thick-film circuit on a ceramic substrate.

This signal is relatively small (at the typical supply voltage of 5 V and a pressure of 100 mmHg it is 2.5 mV, 10 mmHg difference corresponding to a signal change of just 0.25 mV) and is therefore already susceptible to interference. In addition, the signal is transmitted in analog form, and although the connecting cable with a typical length of 2 m is shielded a typical cable shield is not adequate for the sometimes high level of interference. The short cable from a disposable transducer to the connecting cable is normally not shielded, this being so for reasons of cost, since it is used only once, of course. The measured signal according to the standard is therefore very susceptible to interference if the operation is being performed using an electrosurgical appliance or a laser instead of a scalpel.

Finally, the signal is not stored until it is in the patient monitor, i.e. the patient monitor can assess the signal at best using plausibility checks, even if it has its own test intelligence, and can at best reject a signal which is subject to interference, while losing the actual measured data, as is known from DE 198 35 252 A1.

Main section 5 of the standard for invasive blood pressure monitoring appliances DIN EN 60601-2-34 also assumes that the pressure signal on the patient monitor is frequently subject to interference and does not require the signal to be interference-free again until 10 seconds after the interference is cut off. The duration of interference is not limited. This clearly proves that it is prior art that the transmission of the standard-based pressure signal is subject to massive interference, and corresponding failure while a patient is being monitored is also tolerated by the standard.

When using electrical appliances with strong interference fields, such as drills, bone mills, bone saws, electrosurgical appliances, lasers or x ray appliances, as are used particularly for major operations, it is accepted as obviously unavoidable in the case of standard-based measured signal voltages in the prior art that the invasive blood pressure (and possibly also other vital parameters) are not available over relatively long periods and the patient's life is more dependent on the doctor's appraisal of the state of the patient than on continuous monitoring of his state. The downtimes may be considerable in this context. In the case of a hip endoprosthesis operation, the usage times for the bone saw, the drill for preparing the femur and the bone mill for preparing the seat for the artificial socket can easily add up to an hour in which the patient may not be able to be monitored by the patient monitor as expected.

The invention is therefore based on the object of reducing the susceptibility to interference of a measured signal for the invasive blood pressure which has been provided according to standard.

The invention achieves this object by means of a device for transmitting a measured signal for the blood pressure of a patient from a pressure sensor to a display or recording appliance, containing at least a first transmission apparatus (transponder) having at least an analog/digital converter for converting the analog measured signal into digital measured data, having at least a first microprocessor for logically combining the digital measured data with test data and having at least an output stage for transmitting the logically combined digital signal comprising measured data and test data, a second transmission apparatus (transceiver) having a second microprocessor for separately processing measured data and test data, having a comparison apparatus for comparing the test data with reference data and having an output stage for providing the measured data or the measured signal for a display or recording appliance, and a transmission link between the transponder and the transceiver.

The inventive device means that not only can the measured signal be transmitted digitally, which, although it reduces the susceptibility to interference, does not substantially eliminate it, but rather the test data allow the quality of the transmitted data to be checked, uninfluenced by the measured data, by the last link in the transmission chain and hence allow data which is subject to interference to be rejected, and interference-free data to be provided for the display or recording appliance (patient monitor). Even in an environment which is subject to a great deal of interference, it is therefore possible to reduce the downtime for the signal for the patient's blood pressure quite substantially and to limit it to fractions of seconds on the basis of the high possible repetition rate. This allows a significant improvement in the monitoring of a patient's vital parameters.

In one particularly preferred embodiment, the transceiver has an output for transmitting a trigger signal and the transponder has a trigger input for briefly activating its output stage. This allows the output stage of the transponder to be modulated only when the transceiver is requesting a signal, and hence allows the power consumption to be lowered.

To avoid changing the handling of the device over known devices, it is expedient if the transceiver is integrated in a connector for connection to a display or recording appliance.

It is likewise advantageous if the transponder is integrated in the transducer housing of the pressure sensor or is fitted directly on said pressure sensor or is arranged on a mounting support for holding disposable transducers.

A particularly high level of fail safety can be obtained if the transceiver also comprises a current-measuring apparatus for detecting the supply current for operating the transceiver, the transducer and the transponder. This avoids overload cutoff of the blood pressure monitoring by the patient monitor's protective apparatus.

To inform the medical staff in the event of losing the check on the blood pressure parameters on account of prolonged interference, it is advantageous if the transceiver and/or the transponder a signal lamp for displaying a malfunction or relatively long absence of the receipt of interference-free measured data by the transceiver.

In one particularly operationally safe embodiment, the inventive device is characterized in that the output of the transceiver is simultaneously designed to transmit digital reference data or a signal for generating the test data in the transponder. It is thus possible for the data which are to be used as test data to be able to be prescribed by the transceiver's microprocessor and to allow particularly reliable and at the same time simple testing for freedom from interference in the transmission by the transceiver.

The object is also achieved by a method for transmitting a measured signal for the blood pressure of a patient and providing a signal for a display or recording appliance, in which a measured-value sensor (transducer) produces an electrical signal on the basis of the directly or indirectly detected blood pressure, where the signal is converted into a digital measured signal in a first transmission apparatus (transponder) and is logically combined with a test signal, and the logically combined signal is output to a transmission link when requested by a second transmission apparatus (transceiver), where the logically combined signal is received by the transceiver and the test signal is compared with a reference signal, and the digital measured signal is rejected as subject to interference if the test signal differs from the reference signal, or the digital measured signal is output to a display or recording appliance if the test signal does not differ from the reference signal, and where the transceiver requests fresh output of a logically combined signal from the transponder if the received digital measured signal has been rejected as subject to interference or if a predetermined period T1 has elapsed since the last receipt of an interference-free digital measured signal.

The inventive method means that not only is the measured signal transmitted digitally, which, although it reduces the susceptibility to interference, does not substantially eliminate it, but rather testing the data allows the quality of the transmitted data to be checked, uninfluenced by the measured data, by the last link in the transmission chain and hence allows data which is subject to interference to be rejected, and interference-free data to be provided for the display or recording appliance (patient monitor). Even in an environment which is subject to a great deal of interference, it is therefore possible to reduce the downtime for the signal for the patient's blood pressure quite substantially and to limit it to fractions of seconds on the basis of the high possible repetition rate. This allows a significant improvement in the monitoring of a patient's vital parameters.

In this context, it is advantageous for repetition of the data transmission if the digital measured signal is buffer-stored in the transponder until it is replaced by a new digital measured signal.

To avoid unnecessary alarms by the patient monitor, it is expedient if the digital measured signal for output to a display or recording appliance is buffer-stored in the transceiver until it is replaced by a new digital measured signal.

To warn the medical staff in the absence of reliable data for the patient's blood pressure, it is advantageous if an audio of the visual warning signal is output if an interference-free digital measured signal has not been received by the transceiver over a predetermined period T2.

To ensure that the monitor does not erroneously indicate a stable state for the patient even though relevant data are missing, it is expedient if the digital measured signal for output to the display or recording appliance is cut off as soon as an interference-free digital measured signal has not been received by the transceiver over a predetermined period T3.

For full compatibility with monitors based on the current standard, the digital measured signal is converted into an analog measured signal before output to a display or recording appliance.

For simple handling and performance of the method, it is advantageous if the electrical power required for operating at least the transceiver and the transponder is taken from the display or recording appliance.

To avoid unnecessary cutoff of the blood pressure monitoring on faults as a result of overload by the monitor, it is advantageous if the electrical current taken from the display or recording appliance is continually monitored and, if a predetermined threshold value is exceeded, the period T1 is extended, preferably doubled, and/or the operating frequency of the transponder and/or the transceiver is lowered. This allows a substantial reduction in the current drawn by the electronic components of an appropriate device, by approximately 30% per halving of the clock frequency.

To this end, it is expedient if the step of extending the period T1 and/or lowering and/or the operating frequency is repeated until the threshold value for the electrical current has been undershot.

For accurate measured data, it is advantageous if the measured signal is subjected to temperature compensation in the transponder.

It is particularly advantageous if following initialization a reference signal is transmitted from the transceiver to the transponder, the reference signal received by the transponder subsequently being logically combined with the measured signal as a test signal.

The invention is particularly advantageous if it is used for continuously monitoring the blood pressure of a patient.

The invention will be explained in more detail below using an exemplary embodiment of an inventive method and a device which is shown in the drawings, in which:

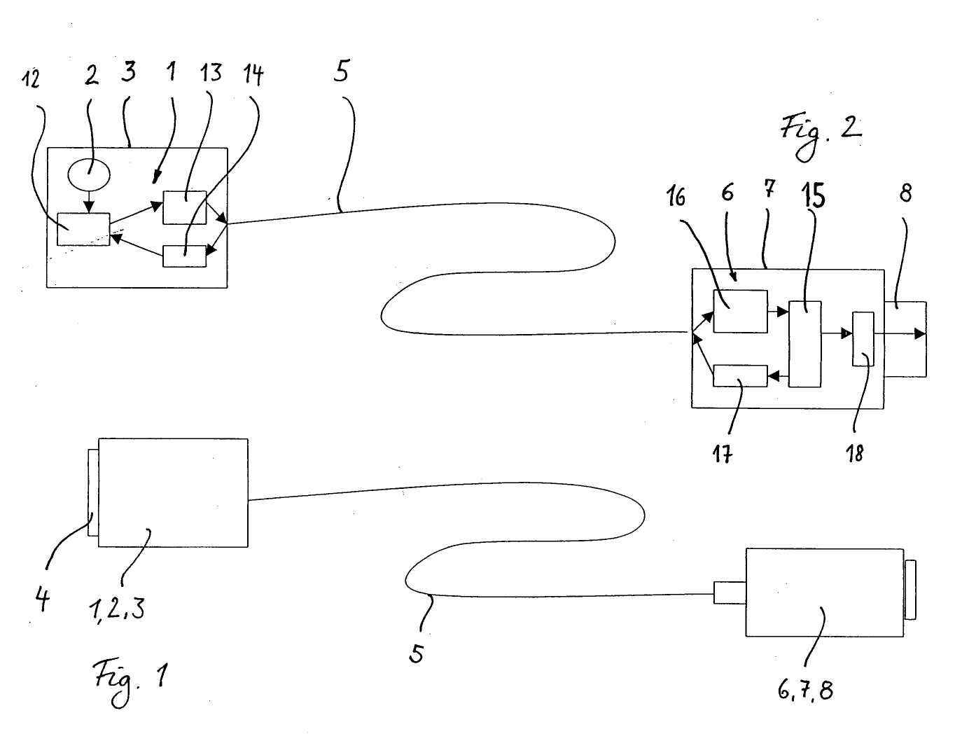

FIG. 1 shows a schematic view of an inventive device,

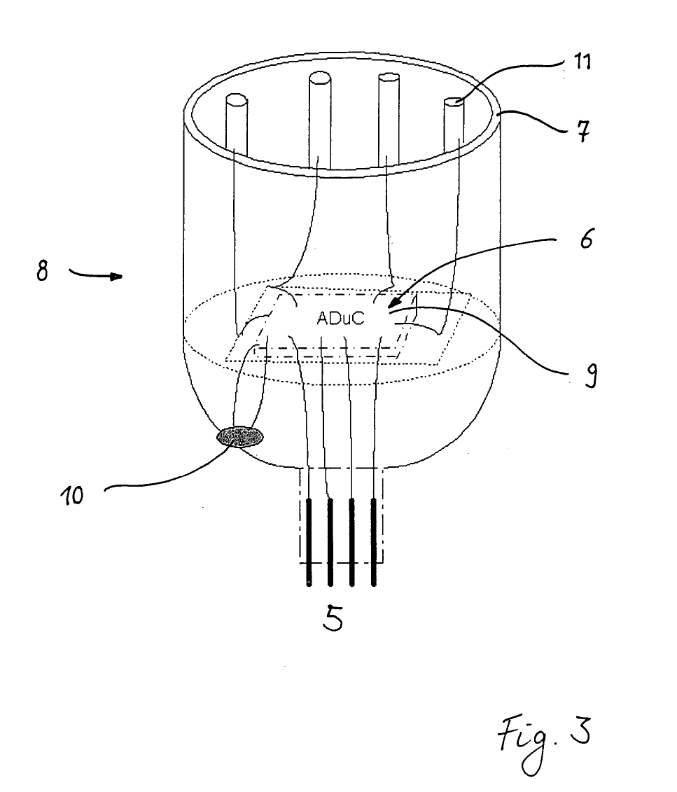

FIG. 2 shows a block diagram of an inventive device, and

FIG. 3 shows a plug connector, with an integrated inventive transceiver.

In the inventive device shown in FIG. 1, a first transmission apparatus, a transponder 1, having a pressure sensor, a transducer 2, is accommodated in a common housing 3. In this arrangement, the housing 3 is designed to be as impermeable as possible to interfering radiation, e.g. a metal housing. The housing 3 has a connecting piece 4 for connection to a pressure dome through which the fluid to be measured, e.g. blood, flows or which is connected to a fluid system having a fluid connection to the blood circulation, e.g. is connected to the column of liquid formed by an infusion solution.

Emerging on one side of the housing 3 is a preferably single-shielded or double-shielded connecting cable 5. The connecting cable 5 may comprise two wire pairs, for example, and forms the transmission link between transponder 1 and transceiver 6.

The transceiver 6 is advantageously integrated in the housing 7 of a connector 8 for connection to a patient monitor as display or recording appliance. A schematic insight into a connector 8 of this kind is provided by FIG. 3. In this case, the housing 7 of the connector 8 accommodates, close to the entry of the connecting cable 5, a microcontroller circuit 9 which essentially comprises a microcontroller having integrated A/D and D/A converters and output drivers. Microcontrollers of this kind are often also called ADuCs. In addition, the housing 7 contains a light-emitting diode 10 which is connected to the microcontroller circuit 9 and is used as visual warning signal in the event of a fault. In addition, the housing 7 of the connector 8 also contains the usual contact pins 11 for the measured signal and the supply voltage.

FIG. 2 schematically shows a block diagram of the device from FIG. 1. In this case, the housing 3 also contains, besides the pressure sensor or transducer 2, the transponder 1. This is formed from a microcontroller circuit which, besides a first microprocessor 12 with an integrated analog/digital converter, also a digital output stage 13 for supplying data to the connecting cable 5, and also preferably an input stage 14 for receiving control signals from the transceiver 6, e.g. trigger signals.

The microcontroller circuit of the transceiver 6 accommodated in the connector housing 7 contains a second microprocessor 15, a digital input stage 16 for receiving the signal transmitted via the connecting cable 5, and also preferably an output stage 17 for transmitting control signals for the transponder 1. In addition, an analog output stage 18 is also for providing the measured data on the connector 8 for a display or recording appliance (patient monitor), not shown. For operation with a monitor based on the currently valid standard, the transceiver 6 also comprises a digital/analog converter, which is used to convert the measured data to the standard's analog format with the sensitivity 5 μV/V/mmHg. For future applications which allow direct digital processing of the data by a patient monitor, a D/A converter stage of this kind can be dispensed with. Expediently, appropriate data buffer stores, known as buffers, are provided in order to keep the respective data available at the outputs beyond a processor cycle too.

All in all, the device is supplied with electrical power from the connection on the patient monitor. The components start to be operated immediately when the connection is made or the monitor is turned on, and separate handling for turn-on operations or the like is not required. Integration of the transceiver into the connector 8 is readily possible, since the circuit has a mass which is only so slight that the mechanical retention forces of the connector 8 are readily sufficient.

The transducer may be a pressure transducer of the usual kind, e.g. piezoresistive. In this case, the signal is a signal voltage proportionally dependent on the effective pressure. The digitally converted pressure values can be stored in a buffer store at a sampling rate of, by way of example, but not necessarily limited thereto, 100 times per second and can be kept there for active checks by the transceiver arranged on the patient monitor side, said transceiver converting back the checked digital information to the standard's (based on DIN EN 60601-2-34) analog signal voltage.

The check by the microcontroller in the transceiver is performed using a protocol which is stored in the microcontroller in nonvolatile form. The transponder's microcontroller holds the information currently being checked until the transceiver's microcontroller notifies it that the information has been received correctly. The check by the transceiver's microcontroller is repeated, upon establishing errors in the control bits, until freedom from error has been established using the control bits. This can happen many hundreds of times on account of the high speed of the microcontrollers, which are typically clocked at hundreds of MHz, and still the pressure can be transmitted every hundredth of a second.

In addition, there may also be a respective light-emitting diode provided as a visual warning signal on the transponder and on the transceiver.

As already mentioned, the signal is converted into a digital measured signal in the first transmission apparatus (transponder 1) and is logically combined with a test signal, and the logically combined signal is output to a transmission link (cable 5) when requested by the second transmission apparatus (transceiver 6). The logically combined signal is received by the transceiver 6 and the test signal is compared with a reference signal. The digital measured signal is rejected as being subject to interference if the test signal differs from the reference signal, or is identified as being erroneous on the basis of a calculation algorithm. If the test signal is identified as being error-free, the digital measured signal is output to a display or recording appliance (patient monitor). The transceiver 6 requests fresh output of a logically combined signal from the transponder 1 if the received digital measured signal has been rejected as being subject to interference or if a predetermined period T1 has elapsed since the last receipt of an interference-free digital measured signal. The digital measured signal is buffer-stored in the transponder until it is replaced by a new digital measured signal. The digital measured signal for output to the monitor is buffer-stored in the transceiver 6 until it is replaced by a new digital measured signal.

An audio of the visual warning signal is output by means of the LED 10, for example, if an interference-free digital measured signal has not been received by the transceiver 6 over a predetermined period T2. The digital measured signal for output to the monitor is cut off if an interference-free digital measured signal has not been received by the transceiver 6 over a predetermined period T3.

Preferably, the output stage 17 of the transceiver 6 is designed such that it is simultaneously designed for transmitting digital reference data or a signal for generating the test data in the transponder 1. Expediently, the reference data which are to be used by the transceiver 6 for checking the data which are to be received are transmitted to the transponder 1 in this way after initialization, e.g. when the supply voltage is applied. When a patient monitor is turned on, there are normally no sources of interference in operation, which means that the reference data can be transmitted to the transponder 1 without error. The reference data are stored in the transponder 1 and are sent to the transceiver 6 in a form logically combined with the measured data as test data or as test signal. In the unusual case in which the transmission of the reference data to the transponder 1 were subject to interference, this would be immediately obvious, since then all the signals received by the transponder 1 would be rejected as being subject to interference, and a signal would not be output to the monitor. This would require the medical staff to reinitialize the device.

Instead of the reference signal or the reference data, it is also possible to transmit an algorithm for generating the test signal to the transponder 1, so that during prolonged operation, for example, a variance in the test signal over time can be obtained or it is possible to encrypt the test signal when a plurality of such devices are being operated simultaneously and in direct proximity.

The electrical current taken from the patient monitor is continually monitored and, if a predetermined threshold value is exceeded, the period T1 is extended, preferably doubled, and/or the operating frequency of the transponder 1 and/or the transceiver 6 is lowered. This step is repeated until the threshold value for the electrical current has been undershot.

Preferably, the measured signal is, subjected to temperature compensation in the transponder 1.

1. A device configured to transmit a signal presenting the blood pressure of a patient from a pressure sensor to a display or recording appliance, the device comprising:
a first transmission apparatus comprising:
an analog/digital converter configured to convert the analog measured signal into digital measured data;
a first microprocessor configured to logically combine the digital measured data with test data to generate a logically combined digital signal;
an output stage configured to transmit the logically combined digital signal;
a second transmission apparatus comprising:
a second microprocessor configured to separately process the digital measured data and the test data;
a comparison apparatus configured to compare the test data with reference data; and
an output stage configured to provide the digital measured data or the analog measured signal a display or recording device; and
a transmission link between the transponder and the transceiver.

2. The device as claimed in claim 1, wherein the second transmission apparatus has an output configured to transmit a trigger signal and the first transmission apparatus includes a trigger input configured to receive the trigger signal, wherein the first transmission apparatus is configured to activate the output stage in response to the received trigger signal.

3. The device as claimed in claim 1, wherein the second transmission apparatus is integrated with a connector configured to connect to a display or recording appliance.

4. The device as claimed in claim 1, wherein the first transmission apparatus is integrated with a housing of a transducer of the pressure sensor or is coupled to said pressure sensor.

5. The device as claimed in claim 1, wherein the first transmission apparatus is arranged on a mounting support configured to hold disposable transducers.

6. The device as claimed in claim 1, wherein the second transmission apparatus also comprises a current-measuring apparatus configured to detect the supply current for operating the second transmission apparatus, the transducer, and the first transmission apparatus.

7. The device as claimed in claim 1, wherein at least one of the first and second transmission apparatus comprise a signal lamp configured to indicate a malfunction or absence of a minimum duration of the receipt of interference-free measured data by the second transmission apparatus.

8. The device as claimed in claim 1, wherein the output of the second transmission apparatus is configured to substantially simultaneously transmit digital reference data and a signal for generating the test data in the first transmission apparatus.

9. A method of transmitting a signal representing a blood pressure of a patient and providing a signal for a display or recording appliance, the method comprising:
producing an analog measured signal with a sensor, the signal being based on the blood pressure, converting the signal into a digital measured signal in a first transmission apparatus;
logically combining the digital measured signal with a test signal to produce a logically combined signal;
outputting the logically combined signal to a transmission link after receiving a request from a second transmission apparatus;
comparing the test signal with a reference signal at the second transmission apparatus;
rejecting the digital measured signal as being subject to interference if the test signal differs from the reference, signal by more than a threshold;
outputting the digital measured signal to a display or recording appliance if the test signal does not differ from the reference, signal by more than the threshold;
requesting fresh output of the logically combined signal from the first transmission apparatus if the digital measured signal has been rejected
requesting fresh output of the logically combined signal from the first transmission apparatus if a predetermined period has elapsed since the last receipt of a non-rejected digital measured signal.

10. The method as claimed in claim 9, wherein the digital measured signal is buffer-stored in the transponder first transmission apparatus until replaced by a fresh digital measured signal.

11. The method as claimed in claim 9, wherein the digital measured signal output to a display or recording appliance is buffer-stored in the first transmission apparatus until replaced by a fresh digital measured signal.

12. The method as claimed in claim 9, wherein an audio signal related to the visual warning signal is output if non-rejected digital measured signal has not been received by the second transmission apparatus after a second predetermined period.

13. The method as claimed in claim 12, wherein the digital measured signal output to the display or recording appliance is terminated if a non-rejected digital measured signal has not been received by the second transmission apparatus after a third predetermined period.

14. The method as claimed in claim 9, further comprising:
converting the digital measured signal into an analog measured signal; and
outputting the analog measured signal to a display or recording appliance.

15. The method as claimed in claim 9, wherein the electrical power required for operating at least the first and second transmission apparatus is taken from sourced by the display or recording appliance.

16. The method as claimed in claim 15, wherein the electrical current sourced by the display or recording appliance is monitored and, if the electrical current exceeds a threshold value, at least one of the following corrective action occurs:
the first predetermined period is extended;
the first predetermined period is doubled;
the operating frequency of the first transmission apparatus is lowered; and
the operating frequency of the second transmission apparatus is lowered.

17. The method as claimed in claim 16, wherein the corrective action is repeated until the electrical current does not exceed the threshold value.

18. The method as claimed in claim 9, wherein the measured signal is subjected to temperature compensation in the first transmission apparatus.

19. The method as claimed in claim 9, wherein following initialization, a reference signal is transmitted from the second transmission apparatus to the first transmission apparatus, and the reference signal is logically combined with the measured signal to generate a test signal.

20. The method of claim 9, further comprising continuously monitoring the blood pressure of a patient.
